(19) United States
(12) Patent Application Publication
(10) Pub. No.: US 2004/0241160 A1
(43) Pub. Date: Dec. 2, 2004

(54) Vegfr-1 antibodies to treat breast cancer

(76) Inventors: Yan Wu, Flemington, NJ (US); Shahin Rafii, Great Neck, NY (US); Larry Witte, Stormville, NY (US)

Correspondence Address: KENYON & KENYON, ONE BROADWAY, NEW YORK, NY, 10004, US

(21) Appl. No.: 10/483,919

(22) PCT Filed: Jul. 15, 2002
(86) PCT No.: PCT/US02/22540

Related U.S. Application Data
(60) Provisional application No. 60/304,751, filed on Jul. 13, 2001.

(51) Int. Cl. A61K 39/395; A61K 31/00

(52) U.S. Cl. 424/143.1; 514/1

(57) ABSTRACT

The present invention is directed to methods of inhibiting tumor cells by administering an antagonist which inhibits the VEGF/VEGFR-1 autocrine loop of tumor cells. Additional antagonists can be added to inhibit endothelial paracrine loop by inhibiting other VEGFRs expressed on endothelial cells, particularly VEGFR-2. Examples of antagonists include antibodies and small molecules. A preferred cancer for treatment is breast cancer.

### FIELD OF THE INVENTION

The present invention is directed to methods of treatment of tumors in mammals with antagonists of VEGF receptors that are expressed on tumor cells. The antagonists are preferably neutralizing antibodies that specifically bind to an extracellular domain of VEGF receptors that are expressed on tumor cells. In particular, the present invention is directed to the treatment of breast cancer via the administration of neutralizing antibodies that specifically bind to an extracellular domain of human VEGFR-1 in amounts effective to reduce tumor growth or size.

### BACKGROUND

Vascular endothelial growth factor (VEGF), placenta-derived growth factor (PlGF), and their receptors VEGFR-1 (Flt-1), VEGFR-2 (KDR, Flk-1), and VEGFR-3 (Flt-4) have been implicated in vasculogenesis, angiogenesis, and tumor growth. VEGF is a homodimeric glycoprotein consisting of two 23 kD) subunits with structural similarity to PDGF. Four different monomeric isoforms of VEGF exist resulting from alternative splicing of mRNA. These include two membrane bound forms (VEGF₂₀₆ and VEGF₁₈₉) and two soluble forms (VEGF₁₆₅ and VEGF₁₂₁). In all human tissues except placenta, VEGF₁₆₅ is the most abundant isoform.

VEGF is a strong inducer of vascular permeability, a stimulator of endothelial cell migration, and an important survival factor for newly formed blood vessels. VEGF is expressed in embryonic tissues (Breier et al., Development (Camb.) 114: 521 (1992)), macrophages, proliferating epidermal keratinocytes during wound healing (Brown et al., J. Exp. Med., 176: 1375 (1992)), and may be responsible for tissue edema associated with inflammation (Ferrara et al., Endocr. Rev. 13: 18 (1992)). In situ hybridization studies have demonstrated high VEGF expression in a number of human tumor lines including glioblastoma multiform, heman-gioblastoma, central nervous system neoplasms and AIDS-associated Kaposi's sarcoma (Plate, K. et al. (1992) Nature 359: 845-848; Plate, K. et al. (1993) Cancer Res. 53: 5822-5827; Berkman, R. et al. (1993) J. Clin. Invest. 91: 153-159; and Nakamura, S. et al. (1992) AIDS Weekly, 13 (1)). High levels of VEGF were also observed in hypoxia induced angiogenesis (Shweiki, D. et al. (1992) Nature 359: 843-845).

The biological response of VEGF is mediated through its high affinity VEGF receptors which are selectively expressed on endothelial cells during embryogenesis (Millauer, B., et al. (1993) Cell 72: 835-846) and during tumor formation. VEGF receptors typically are class III receptor-type tyrosine kinases characterized by having several, typically 5 or 7, immunoglobulin-like loops in their amino-terminal extracellular receptor ligand-binding domains (Kaipainen et al., J. Exp. Med. 178: 2077-2088 (1993)). The other two regions include a transmembrane region and a carboxy-terminal intracellular catalytic domain interrupted by an insertion of hydrophilic interkinase sequences of variable lengths, called the kinase insert domain (Terman et al., Oncogene 6: 1677-1683 (1991)).

VEGF receptors include VEGF receptor 1 (VEGFR-1, also called fins-like tyrosine kinase receptor, or Flt-1), sequenced by Shibuya M. et al., Oncogene 5, 519-524 (1990); and VEGF receptor 2 (VEGFR-2). The human form of VEGFR-2 is also called kinase insert domain-containing receptor (KDR) and is described in PCT/US92/01300, filed Feb. 20, 1992, and in Terman et al., Oncogene 6: 1677-1683 (1991). The murine form of VEGFR-2 is also called FLK-1 and was sequenced by Matthews W. et al. Proc. Natl. Acad. Sci. USA, 88: 9026-9030 (1991).

Release of VEGF by a tumor mass stimulates angiogenesis in adjacent endothelial cells. When VEGF is expressed by the tumor mass, endothelial cells closely adjacent to the VEGF+tumor cells will up-regulate expression of VEGF receptor molecules e.g., VEGFR-1 and VEGFR-2. Upon binding of their ligand, these receptors dimerize and transduce an intracellular signal through tyrosine phosphorylation. VEGF plays a crucial role for the vascularization of a wide range of tumors including breast cancers, ovarian tumors, brain tumors, kidney and bladder carcinomas, adenocarcinomas, malignant gliomas and luekemias. Tumors produce ample amounts of VEGF, which stimulates the proliferation and migration of endothelial cells (ECs), thereby inducing tumor vascularization by a paracrine mechanism.

Placenta-derived growth factor (PlGF), another natural specific ligand for VEGFR-1 (Flt-l), which is produced in large amounts by villous cytotrophoblast, sincytiotrophoblast and extravillous trophoblast, is a member of the VEGF family. PlGF is a dimeric secreted factor which shares close amino acid homology to VEGF. Some of the biological effects of VEGF and PlGF are also similar, including stimulation of endothelial cell migration. PlGF and VEGF, thus appear capable of acting in unison on both myelomonocytic and endothelial lineage cells.

The administration of neutralizing antibodies and other molecules that block signaling by VEGF receptors expressed on vascular endothelial cells is known to reduce tumor growth by blocking angiogenesis through an endothelial-dependent paracrine loop. One advantage of blocking the VEGF receptor, as opposed to blocking the VEGF ligand to inhibit angiogenesis, and thereby inhibit pathological conditions such as tumor growth, is that fewer antibodies may be needed to achieve such inhibition. Furthermore, receptor expression levels may be more constant than those of the environmentally induced ligand. See, U.S. Pat. Nos. 5,804,301; 5,874,542; 5,861,499; and 5,955,311.

Certain tumor cells not only produce VEGF, but may also have acquired the capacity to express functional VEGF receptors (VEGFR), which results in the generation of an endothelial-independent autocrine loop to support tumor growth. The present inventors have recently provided the first demonstration that a VEGF/human VEGFR-2 autocrine loop mediates leukemic cell survival and migration in vivo. Dias et al., “Autocrine stimulation of VEGFR-2 activates human leukemic cell growth and migration,” J. Clin. Invest. 106: 511-521 (2000). Similarly, VEGF production and VEGFR expression also have been reported for some solid tumor cell lines in vitro. (See Tohoku, Sato, J. Exp. Med., 185(3): 173-84 (1998); Nippon, Sanka Fujinka Gakkai Zasshi,:47(2): 133-40 (1995); and Ferrer, FA, Urology, 54(3):567-72 (1999)). However whether VEGFRs expressed on solid tumor cells are functional and convey mitogenic or other signals has not been demonstrated.

### SUMMARY OF THE INVENTION

The present invention provides a method for treatment of a tumor in a mammal comprising treating the mammal having such a tumor with an antagonist of a VEGF receptor that is expressed on a tumor cell, wherein said VEGF receptor is selected from the group consisting of human VEGFR-1, VEGFR-2, VEGFR-3, neuropilin, and their non-human homologs (such as FLK-1); and wherein said antagonist is administered in an amount effective to reduce tumor growth or size. Preferably, the antagonist is a neutralizing antibody that specifically binds to an extracellular domain of a VEGF receptor that is expressed on a tumor cell, and inhibits autocrine stimulation. Examples of solid tumors which may be treated with the methods and antibodies of the present invention include breast carcinoma, lung carcinoma, colorectal carcinoma, pancreatic carcinoma, glioma, and lymphoma; examples of liquid tumors include leukemia.

In a preferred embodiment, the present invention provides a method for treatment of breast cancer in a mammal comprising treating the mammal having breast cancer with a neutralizing antibody that specifically binds to an extracellular domain of human VEGFR-1, wherein said antibody is administered in an amount effective to reduce tumor growth or size.

In another embodiment, a second VEGF receptor antagonist is also administered. The second antagonist is preferably an antibody against VEGF receptors expressed on tumor-associated vascular endothelial cells, resulting in inhibition of endothelial dependent paracrine loop.

### BRIEF DESCRIPTION OF THE DRAWINGS

FIG. 1 presents an immunoblot for pErk ½ expression in DU4475 human breast cancer cells treated with growth factors, as detailed in Example 1.

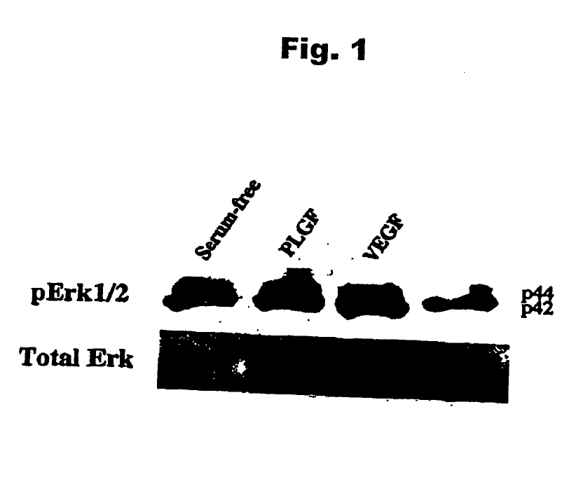

FIG. 2 is a chart showing a densiometry analysis of the blot of FIG. 1.

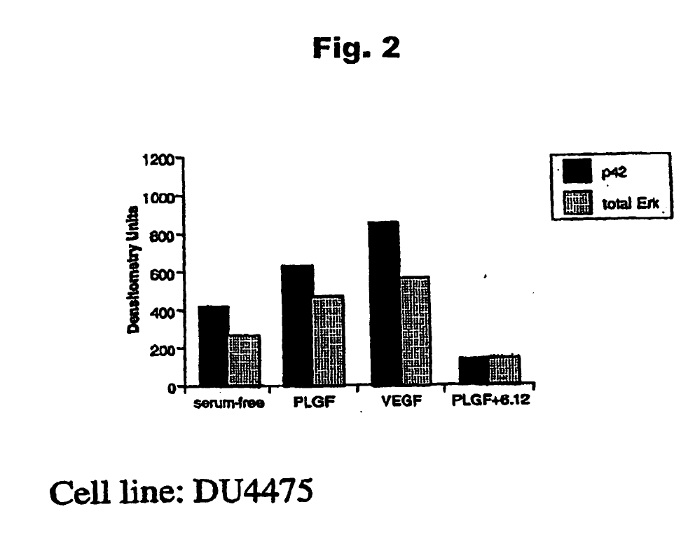

FIG. 3 is a chart showing the results of treatment of NOD-SCID mice inoculated with DU4475 human breast cancer cells with combinations of antibodies, as detailed in Example 2.

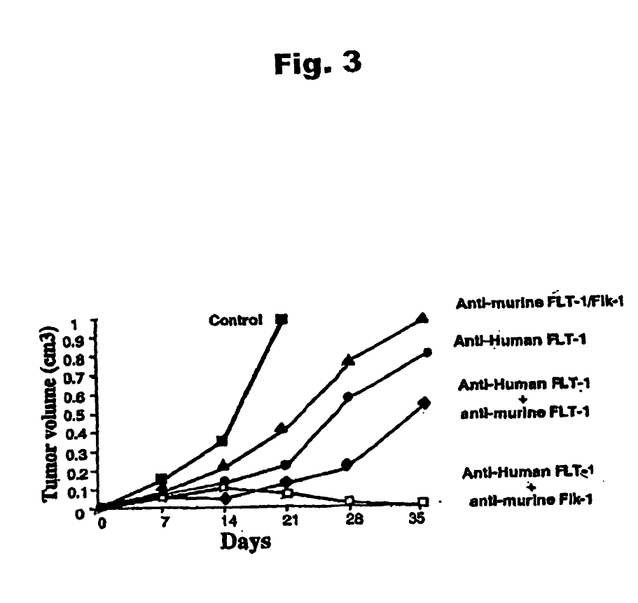

FIG. 4 presents photographs of tissues from NOD-SCID mice inoculated with DU4475 human breast cancer cells after treatment with combinations of antibodies, as detailed in Example 2. The tissues are stained for morphological evaluation.

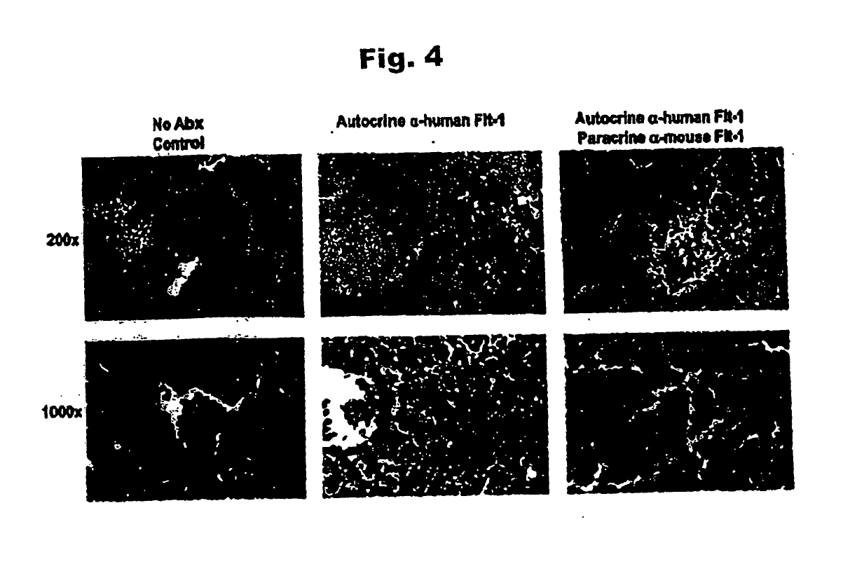

### DETAILED DESCRIPTION

Functional VEGF receptors expressed on tumor cells, and antibodies that bind to such VEGF receptors, as well as small molecules that block the activity of such receptors, are useful for treating tumors by directly inhibiting growth of tumor cells. Therefore, inhibition of tumor cell growth is not dependent upon blocking angiogenesis.

The present invention provides methods and compositions for treating solid tumors, wherein antagonists of VEGF receptors expressed on the tumor cells are administered to a mammal having such a tumor. Preferably, the antagonist is a neutralizing antibody that binds to VEGF receptors expressed on solid tumor cells, and inhibits autocrine loop. The antagonist may also be a small molecule.

The present invention provides antibodies for treating tumors, wherein antibodies bind to and inhibits the activity of VEGF receptors on the tumor cells.

Tumors, the growth of which may be reduced using the methods of the present invention, include tumors that express VEGF receptors. Examples of tumors include breast carcinoma, lung carcinoma, colorectal carcinoma, pancreatic carcinoma, glioma, lymphoma, and leukemias.

The present invention provides methods for identifying antibodies useful for treating a given tumor type, as well as methods for identifying antibodies useful for treating a tumor of a specific patient.

Tumor cells, which may be from established tumor cell lines, from tissue biopsies, from the blood, or from other appropriate sources may be assayed to determine whether and which functional VEGF receptors are expressed thereon. The presence of VEGF receptors may be detected by imunohistochemical, flow cytometry, ELISA assays, and other known methods, coupled with the guidance provided herein. For VEGF receptors found to be present, cells may be tested for receptor function by exposing them to agonist ligands of VEGF receptors and determining whether receptor phosphorylation occurs. Methods of determining receptor phosphorylation are well known in the art and include, for example, measurement of phosphotyrosine with monoclonal antibodies or radioactive labels. Other markers of receptor function, such as cell proliferation and activation of cell signaling pathways known to be activated by the VEGF receptor of interest, may also be tested. Appropriate markers for functionality will vary depending on the VEGF receptor of interest.

The present invention provides antibodies that are capable of binding specifically to the extracellular domain of a VEGF receptor expressed on a tumor cell. VEGF receptors include human VEGFR-1, VEGFR-2, VEGFR-3, and neuropilin, and their non-human homologs (such as FLK-1). An extracellular domain of a VEGF receptor as herein defined includes the ligand-binding domain of the extracellular portion of the receptor, as well as extracellular portions that are involved in dimerization and overlapping epitopes. When bound to the extracellular domain of a VEGF receptor, the antibodies effectively block receptor activation and/or interfere with receptor dimerization. As a result of such binding, the antibodies neutralize activation of the VEGF receptor. Neutralizing a receptor means diminishing and/or inactivating the intrinsic ability of the receptor to transduce a signal. A reliable assay for VEGF receptor neutralization is inhibition of receptor phosphorylation. Methods of determining receptor phosphorylation are well known in the art and include, for example, measurement of phosphotyrosine with monoclonal antibodies or radioactive labels.

In a preferred embodiment, an antibody of the present invention binds to human VEGFR-1 and blocks VEGF binding and/or PlGF binding to human VEGFR-1. Mab 6.12 is an example of an antibody that binds to soluble and cell surface-expressed human VEGFR-1. A hybridoma cell line producing Mab 6.12, has been deposited as ATCC number PTA-3344. The deposit was made under the provisions of the Budapest Treaty on the International Recognition of the Deposit of Microorganisms for the Purposes of Patent Procedure and the regulations thereunder (Budapest Treaty). This assures maintenance of a viable culture for 30 years from date of deposit. The organisms will be made available by ATCC under the terms of the Budapest Treaty, and subject to an agreement between Applicants and ATCC which assures unrestricted availability upon issuance of the pertinent U.S. patent. Availability of the deposited strains is not to be construed as a license to practice the invention in contravention of the rights granted under the authority of any government in accordance with its patent laws.

In addition to the aforementioned antibodies, other anti-VEGF neutralizing antibodies (e.g., antibodies to VEGFR-1, VEGFR-2, VEGFR-3 and neuropilin) may readily be produced using art-known methods combined with the guidance provided herein. The antibodies of the present invention may bind to VEGF receptors with an affinity comparable to, or greater than, that of the natural ligand.

Antibodies that are useful in the present invention include polyclonal and monoclonal antibodies. Both polyclonal and monoclonal antibodies may be produced by methods known in the art. Methods for producing monoclonal antibodies include the immunological method described by Kohler and Milstein in Nature 256, 495-497 (1975) and Campbell in “Monoclonal Antibody Technology, The Production and Characterization of Rodent and Human Hybridomas” in Burdon et al., Eds, Laboratory Techniques in Biochemistry and Molecular Biology, Volume 13, Elsevier Science Publishers, Amsterdam (1985); as well as by the recombinant DNA method described by Huse et al. in Science 246, 1275-1281 (1989).

Chimeric, humanized, and fully human antibodies are also useful in the present invention. Useful chimeric antibodies include chimeric antibodies comprising an amino acid sequence of a human antibody constant region and an amino acid sequence of a non-human antibody variable region. However, chimeric antibodies comprising an amino acid sequence of a non-human antibody constant region and an amino acid sequence of a non-human antibody variable region may also be useful. The non-human variable region of chimeric antibodies may be murine. Useful humanized antibodies include humanized antibodies comprising amino acid sequences of variable framework and constant regions from a human antibody. The amino acid sequence of the hypervariable region of humanized antibodies may be murine.

Chimeric, humanized, or fully human antibodies may be produced by art-known methods, including phage display. (See, e.g., Jones, P. T. et al., (1996) Nature 321, 522-525; Riechman, L. et al., (1988) Nature 332, 323-327; U.S. Pat. No. 5,530,101 to Queen et al.; Kabat, E. A., et al. (1991) Sequences of Proteins of Immunological Interest. 5th ed. National Center for Biotechnology Information, National Institutes of Health, Bethesda, Md.; Queen, C. et al., (1989) Proc. Natl. Acad. Sci. USA 86, 10029-10033; McCafferty et al. (1990) Nature 348, 552-554; Aujame et al. (1997) Human Antibodies 8, 155-168; and Griffiths et al. (1994) EMBO J. 13, 3245-3260). Human antibodies may also be produced from transgenic animals (reviewed in Brüggemann and Taussig (1997) Curr. Opin. Biotechnol. 8, 455-458; see also, e.g., Wagner et al. (1994) Eur. J. Immunol. 42,2672-2681; Green et al. (1994) Nature Genet. 7, 13-21).

Antibodies of the invention also include antibodies that have been made less immunogenic by replacing surface-exposed residues to make the antibody appear as self to the immune system. (See, e.g., Padlan, E. A. (1991) Mol. Immunol. 28, 489-498; Roguska et al. (1994) Proc. Natl. Acad. Sci. USA 91, 969-973).

Antibodies useful in the present invention also include those for which binding characteristics have been improved by direct mutation or by methods of affinity maturation. (See, e.g., Yang et al. (1995) J. Mol. Bio. 254, 392-403; Hawkins et al. (1992) J. Mol. Bio. 226,889-896; Low et al. (1996) J. Mol. Bio. 250, 359-368).

Functional fragments and equivalents of antibodies are also useful in the invention, where such fragments and equivalents have the same binding characteristics as, or that have binding characteristics comparable to, those of the corresponding whole antibody. Such fragments may contain one or both Fab fragments or the F(ab′)2 fragment. Such fragments may also contain single-chain fragment variable region antibodies, i.e., scFv. Fragments may be produced by art-known methods. (See, e.g. Lamoyi et al, Journal of Immunological Methods 56, 235-243 (1983); and Parham, Journal of Immunology 131, 2895-2902 (1983)).

In another aspect of the invention, the antibodies can be chemically or biosynthetically linked to anti-tumor agents or detectable signal-producing agents. The invention further contemplates antibodies to which target or reporter moieties are linked.

In addition to antibodies and their functional equivalents, other biological antagonists that may be used include proteins, peptides, or nucleic acid molecules, including antisense oligonucleotides, which inhibit growth of tumor cells expressing VEGF receptors by blocking receptor activation, for example.

Other useful antagonists may be small molecules, which may be organic or inorganic, and which inhibit growth of tumor cells expressing VEGF receptors by blocking receptor activation, for example. Typically such small molecules have molecular weights less than 500, more typically less than 450. Most typically, the small molecules are organic molecules that usually comprise carbon, hydrogen, and optionally oxygen and/or sulfur atoms.

In another embodiment of the invention, a second VEGF receptor antagonist is administered in addition to an antagonist to a VEGF receptor expressed on tumor cells, to inhibit endothelial dependent paracrine loop. If a VEGFR-1 antagonist is used as a first antagonist, then the second antagonist preferably inhibits another VEGF receptor. In such a case, the VEGFR-1 antagonist inhibits both autocrine and paracrine loops associated with VEGFR-1, thus making it unnecessary to add another VEGFR-1 antagonist. The second antagonist is preferably a neutralizing antibody and preferably targets a VEGF receptor or other growth factor receptor expressed on tumor vasculature. Preferably, the second antagonist inhibits angiogenesis.

An example of such a second antagonist is an antibody that binds to human VEGFR-2 (KDR) and blocks VEGF binding to KDR. scFv p1C11 was produced from a mouse scFv phage display library. (Zhu et al., 1998). p1C11 blocks VEGF-KDR interaction and inhibits VEGF-stimulated receptor phosphorylation and mitogenesis of human vascular endothelial cells (HUVEC). This scFv binds both soluble KDR and cell surface-expressed KDR on HUVEC, for example, with high affinity (K_{d}=2.1nM). DC101 is a rat monoclonal antibody that binds to a neutralized mouse VEGFR-2. A hybridoma cell line producing DC101 was deposited as ATCC Accession No. ATCC HB 11534 on Jan. 26, 1994. Another example of such antibody is MF1, an antagonist of murine VEGFR-1, which inhibits endothelial dependent paracrine and autocrine loop in mice. Yan Wu et al., “Inhibition of Tumor Growth and Angiogenesis in animal models by a neutralizing anti-VEGFR 1 monoclonal antibody”, ImClone Systems Incorporated, New York.

When administering an antibody such as Antibody 6.12 to a human, the Antibody by itself inhibits both autocrine and paracrine loops; the Antibody inhibits VEGFR-1 regardless of the location of the receptor on a tumor cell or endothelial cell. With regard to Example 2, the model involves a human tumor in a mouse, where the endothelial cells are of murine origin. Antibody 6.12 is specific for human VEGFR-1, and thus only inhibits the autocrine loop of the human cancer cells in the mouse model, and not mouse endothelial cells. The paracrine stimulation of mouse endothelial cells thus is unaffected by Antibody 6.12 in the model. MF1 is mouse specific, and inhibits mouse endothelial cells, but not human tumors.

In yet another aspect of the present invention, a patient having a tumor that is substantially not vascularized or not yet vascularized is treated with an antagonist of a VEGF receptor that is expressed on the tumor cells. An example of such a patient is one having a tumor that is undergoing metastasis, wherein the metastases are not yet vascularized. In a preferred embodiment, the patient has metastatic breast cancer and the antagonist is a neutralizing antibody against VEGFR-1.

The antagonists of the present invention may also be used in combined treatment methods. The antibodies and small molecules can be administered along with an anti-neoplastic agent such as a chemotherapeutic agent, a radioisotope, or radiation treatment. Suitable chemotherapeutic agents are known to those skilled in the art and include anthracyclines (e.g. daunomycin and doxorubicin), methotrexate, vindesine, neocarzinostatin, cis-platinum, chlorambucil, cytosine arabinoside, irinotecan, 5-fluorouridine, melphalan, ricin, calicheamicin, taxol, gemcitibine, fluorouracil, paclitaxel, docetaxel, leucovorin and novelbine. The antagonists of the present invention may be administered in combination with other treatment regimes. For example, antibodies and/or small molecules of the invention can be administered with external treatment, e.g., external beam radiation.

It is understood that antibodies and/or small molecules of the invention, where used in the human body for the purpose of diagnosis or treatment, will be administered in the form of a composition additionally comprising a pharmaceutically-acceptable carrier. Suitable pharmaceutically acceptable carriers include, for example, one or more of water, saline, phosphate buffered saline, dextrose, glycerol, ethanol and the like, as well as combinations thereof. Pharmaceutically acceptable carriers may further comprise minor amounts of auxiliary substances such as wetting or emulsifying agents, preservatives or buffers, which enhance the shelf life or effectiveness of the binding proteins.

Methods of administration to a mammal, including humans, include but are not limited to oral, intravenous, intraperitoneal, intracerebrospinal, subcutaneous, intrathecal, intramuscular, inhalation, or topical administration.

The compositions of this invention may be in a variety of forms. These include, for example, solid, semi-solid and liquid dosage forms, such as tablets, pills, powders, liquid solutions, dispersions or suspensions, liposomes, suppositories, injectable and infusible solutions. The preferred form depends on the intended mode of administration and therapeutic application. The preferred compositions are in the form of injectable or infusible solutions.

Effective dosages and scheduling regimens of administration of antibodies according to the present invention can be determined by the skilled practitioner using art-known methods, such as clinical trials and animal studies. Concentrations of the administered substances will vary depending upon the therapeutic or preventive purpose.

In embodiments where two antagonists are co-administered, or where an antagonist is combined with another mode of treatment, each of the treatments may, if desired, be administered in a dosage that is smaller or less frequent than the dosage which would be administered were each treatment administered independently of the other.

All citations throughout the specification and the references cited therein are hereby expressly incorporated by reference.

The Examples that follow are set forth to aid in understanding the invention but are not intended to, and should not be construed to, limit its scope in any way. The Examples do not include detailed descriptions of conventional methods, such as those employed in the construction of vectors and plasmids, the insertion of genes encoding polypepfides into such vectors and plasmids, or the introduction of plasmids into host cells. Such methods are well known to those of ordinary skill in the art and are described in numerous publications including Sambrook, J., Fritsch, E. F. and Maniatis, T. (1989) Molecular Cloning: A Laboratory Manual, 2nd edition, Cold Spring Harbor Laboratory Press.

### EXAMPLES

### Example 1

### Breast Carcinoma Cells Express Functional VEGFR-1 (Flt-1)

The present experiments show that breast cancer cells express functional VEGFR-1. Two human cell lines DU-4475 (ER negative) and MCF-7 (ER positive) were studied extensively. Both cell lines are VEGFR-1 positive. VEGFR-1 expressed by these breast cancer cells is functional, as determined by PlGF-induced receptor tyrosine phosphorylation and activation of the MAP kinase (Erkl/2) pathway. Activation of the MAP kinase pathway by PlGF or VEGF, ultimately leads to increased cell proliferation in vitro. Furthermore, DU-4475 and MCF-7 do not express VEGFR-2, and are therefore growth inhibited only by neutralizing mAb to VEGFR-1 (6.12—blocks only human VEGFR-1).

Immunohistochemical Analysis of Human Breast Carcinomas

Formalin-fixed, paraffin-embedded tissue of 16 human ductal breast carcinoma biopsies were evaluated for VEGFR-1, human VEGFR-2, VEGF and von Willebrand factor (VWF) immunoreactivity, following conventional protocols. The antibodies used were mAb to human VEGFR-1 (FB5); human VEGFR-2 (6.64); VEGF polyclonal antibody, and vWF polyclonal antibody (Zymed Laboratories Inc., South San Francisco, Calif., USA). Secondary peroxidase-labeled antibodies were used at a 1:6000 dilution. The peroxidase reaction was developed with a diaminobenzidine substract and slides were counterstained with hematoxylin and eosin. All sections were observed under a light microscope.

Cell Culture

The human breast cancer cell lines DU4475, MCF-7, T-47D and MDA-MB-231 were obtained from ATCC (Manassas, Va., USA). DU4475 cells were grown in suspension, whereas MCF-7, T-47D and MDA-MB-231 cells were grown as subconfluent monolayer cultures in RPMI 1640 (Bio Whittaker Inc., Walkersville, Md., USA) supplemented with 10% fetal bovine serum, penicillin (100 U/ml), streptomycin (100 μg/ml), fungizone (0.25 μg/ml) and L-glutamine (0.584 mg/ml) (Gibco BRL, Rockville, Md., USA). HUVECs were obtained and cultured as previously described (J Clin Invest. 1973, 52(11): 2745-56). Cells were kept in a humidified incubator under 5% CO₂ at 37° C.

RNA Extraction, cDNA Synthesis and RT-PCR

Total RNA was isolated using Trizol (Gibco BRL, Rockville, Md., USA), following the manufacturer's instructions. First-strand cDNA was subsequently synthesized using SuperScript II reverse transcriptase, according to manufacturer's protocol (Amersham Pharmacia Biotech, Piscataway, N.J. USA). PCR was performed using Advantage 2 polymerase mix (Clontech Laboratories Inc., Palo Alto, Calif., USA). Amplification conditions were as follows: a precycle of 5 minutes at 94° C., 45 seconds at 63° C., and 45 seconds at 72° C.; followed by 35 cycles at: 94 minute, 63° C. for 45 seconds, 72° C. for 2 minutes and a 7 minute extension at 72° C. Primers used for the PCR:

VEGFR-1 (forward: ATTTGTGATTTTGGCCTTGC; reverse:
CAGGCTCATGAACTTGAAAGC); human VEGFR-2 (forward:
GTGACCAACATGGAGTCGTG; reverse: CCAGAGATTCCATGCCACTT) VEGF
(forward: CGAAGTGGTGAAGTTCATGGATG; reverse:
TTCTGTATCAGTCTTTCCTGGTGAG); PIGF (forward:
CGCTGGAGAGGCTGGTGG; reverse: GAACGGATCTTTAGGAGCTG)) and Beta-
actin (forward: TCATGTTTGAGACCTTCAA, reverse:
GTCTTTGCGGATGTCCACG).

Oligonucleotide primers designed were used to amplify 3 of the VEGF splicing variants (variants 121, 165, 189).

Flow Cytometry Analysis

For identification of VEGFR-1/Flt-1⁺and VEGFR-2/KDR⁺cells, DU4475, MCF-7, T-47D and MDA-MB-231 cells were incubated with 2 μl of FITC-labeled high-affinity, mAb to Flt-1 (clone FB5), or with an unconjugated mAb to KDR (clone 6.64), for 20 minutes. A secondary PE-labeled Ab (Kirkegaard & Perry Laboratories, Gaithersburg, Md., USA) was subsequently added to the latter for 20 minutes. The number of positive cells for VEGFR-1 or human VEGFR-2 was determined using a Coulter Elite flow cytometer (COULTER, Hialeah, Fla., USA) and compared to an immunoglobulin G isotype control (FITC; Immunotech, Marceille, France). Nonviable cells were identified by propidium iodide (PI) staining.

Quantification of VEGF and PlGF Levels in Cell Culture Supernatants

ELISA kits specific for human VEGF₁₆₅ or PlGF (R&D Systems Inc., Minneapolis, Minn., USA) were used to determine VEGF and PlGF production in human breast cancer cells. DU4475, MCF-7, T-47D and MDA-MB-231 cell lines were seeded in 6-well plates at a density of 10⁶ cells/well. Cells were cultured in serum-free conditions, and supernatants were collected after 48 hours. These were used without further dilution. Each sample was measured in duplicate.

Cell Proliferation Assays

Proliferation of DU4475 cells was determined by counting the number of viable cells, using the Trypan blue exclusion test, and by using the BrdU incorporation assay.

For the trypan blue exclusion test, cells were seeded at a density of 2.5×10⁵/well into 12-well plates in serum-free RPMI. The cultures were treated every 24 hours with: 50 ng/ml PlGF, 20 ng/ml VEGF (R&D Systems Inc., Minneapolis, Minn., USA), 1 μg/ml of the mAb against human VEGFR-1 (clone 6.12) or untreated, for 48 at 37° C. Viable cells were counted in triplicate using a hemacytometer. Each experiment was done in triplicate.

For the BrdU incorporation assay, 5×10³ cells were plated in 96-well plates for 48 hours, in the following conditions: serum-free, VEGF (50 ng/mL), PlGF (100 ng/mL), clone 6.12 mAb against VEGF-1 (1 μg/ml) and co-incubation with 6.12 and PlGF. BrdU was added to the cultures for the last 24 hours. Incorporated BrdU was quantified using an ELISA kit (Roche Diagnostics, Mannheim, Germany), following the manufacturer's protocol.

VEGFR-1 Phosphorylation Assay

For receptor phosphorylation assay, DU4475 cells were seeded in 12 well-plates (5×10⁵ cells/well) and kept in RPMI serum-free medium for 18 hours. After replacing the culture medium, the cells were treated with VEGF (50 ng/ml), PlGF (100 ng/ml) for 10 minutes or co-incubated with mAbs to human VEGFR-1 and human VEGFR-2 (clone 6.12 and IMC-1C11, respectively), for 1 hour and PlGF for 10 minutes, at 37° C. After stimulation, total protein extracts were obtained by lysing cells in cold RIPA buffer (50 mM Tris, 5 mM EDTA, 1% Triton X-114, 0.4% sodium cacodylate, and 150 mM NaCl), in the presence of protease inhibitors (1 mg/mL aprotinin, 10 mg/mL leupeptin, 1 mM glycerophosphate, 1 mM sodium orthovanadate, and 1 mM PMSF), for 30 minutes at 4° C. Supernatants from protein extracts were immunoprecipitated overnight at 4° C. in the presence of an anti-phosphotyrosine antibody (PY20) and protein-G agarose beads (Santa Cruz Biotechnology Inc., Santa Cruz, Calif., USA), to precipitate phosphorylated proteins. The immunoprecipitates were resuspended in loading buffer, and fractionated under reducing conditions (in the presence of β-mercaptoethanol) by SDS-PAGE using 7.5% polyacrylamide gels. Proteins were subsequently electroblotted onto a nitrocellulose membrane. Blots were blocked in 1% BSA/PBS—1% Tween-20, for 1 hour at room temperature and then incubated with primary and secondary antibodies. Mouse monoclonal antibody anti-VEGFR-1 (R&D Systems Inc., Minneapolis, Minn., USA) was used at a concentration of 1μg/mL, and secondary anti-mouse IgG-HRP (Santa Cruz Biotechnology Inc., Santa Cruz, Calif., USA) was used at 1:6000. The ECL chemiluminescence detection system and ECL film (Amersham Pharmacia Biotech, Piscataway, N.J., USA) were used for the detection of proteins on the nitrocellulose blots.

MAP Kinase Pathways Activation Through Flt-1

To evaluate MAPK phosphorylation, DU4475 cells were seeded in 12 well-plates (5×10⁵ cells/well) in serum-free RPMI for 18 hours. The cells were then washed 3 times with cold PBS, and treated with or without growth factors (VEGF, 50 ng/mL; PlGF, 100 ng/mL) for 10 minutes or preincubated with clone 6.12 for 1 hour and then stimulated with PlGF for 10 minutes. Cells were also treated with p42/p44 and p38 inhibitors, PD98059 (30 μM) and SB203580 (20 μM) respectively, for 1 hour and stimulated with PlGF for 10 minutes. Cell lysis and protein isolation were performed as described above. Proteins were subjected to a 7.5% SDS-PAGE and electroblotted onto nitrocellulose membranes. Following transfer, the membranes were immunoblotted with an antibody against p42/p44 MAP kinases (Thr202/Tyr204) (Santa Cruz Biotechnology Inc., Santa Cruz, Calif., USA) and p38 MAP kinase (Thr180/Tyr182), at a concentration of 1 μg/mL, followed by incubation with a secondary anti-mouse IgG-HRP (1:5000). To ensure equal loading of samples, membranes were stripped and reprobed with anti-p42/p44 (Santa Cruz Biotechnology Inc., Santa Cruz, Calif., USA) or anti-p38 antibodies.

Akt Phosphorylation Assay

DU4475 cells were seeded in 12 well-plates (5×10⁵ cells/well) in serum-free RPMI for 18 hours. The cells were then washed 3 times with cold PBS, treated with or without growth factors or anti-human VEGFR-1 mAb as indicated above, and also co-incubated with the PI3-kinase inhibitor wortmannin (30nM) for 1 hour and PlGF for 10 minutes. Cell lysis, protein isolation, SDS-PAGE and electroblot into nitrocellulose membranes were performed as described previously. Levels of Akt phosphorylation (Ser473) were detected using a primary mouse polyclonal anti-phospho-Akt antibody (Santa Cruz Biotechnology Inc., Santa Cruz, Calif., USA), at a concentration of 1 μg/mL, followed by incubation with a secondary anti-mouse IgG-HRP (1:5000). To confirm equivalent protein loading, membranes were stripped and reprobed with anti-Akt antibodies (Santa Cruz Biotechnology Inc., Santa Cruz, Calif., USA).

Analysis of Apoptosis in Breast Cancer Cell Lines

DU4475 cells were seeded in 12 well-plates (5×10⁵ cells/well), and kept for 48 hours under the following conditions: serum-free RPMI 1640, RPMI with 10% FCS, clone 6.12 (2 μg/mL), clone 6.12 (10 μg/mL) and 4% paraformaldehyde (positive control). Cells were harvested and stained by fluorescein isothiocyanate-conjugated annexin V and by PI, following the manufacturer's instructions (Immunotech, Marceille, France).

Results were analyzed using a Coulter Elite flow cytometer (COULTER, Hialeah, Fla., USA). Cells which were double positive for FITC-labeled annexin V, and PI were considered apoptotic.

In vivo Effects of VEGFR-1 mAbs in the Growth of Established DU4475 Breast Tumors

To evaluate the effect of VEGFR-1 Abs against fully established tumors, DU4475 human breast tumor cells (1×10⁶) were injected subcutaneous into athymic nude mice (Jackson Labs, Bar Harbor, Me., USA). Mice were divided in groups of 16 animals each and tumors were allowed to grow up to approximately 20, 120 and 400 mm³ in size. Treated animals received intraperitoneal injections of 1000 μg of: anti-mouse VEGFR-1 mAb (mF1), anti-human VEGFR -1 mAb (6.12), or the combination of both, every 3 days. The control group was injected with PBS. Tumors were measured twice a week for 42 days. Tumor tissues were taken for histological examination on days 14, 30 and at the end of the experiment after antibodies treatment.

### Example 2

### mAb to VEGFR-1 Blocks Breast Cancer Gowth in vivo

Subcutaneous inoculation into NOD-SCID mice of DU-4475 human breast carcinoma cells resulted in the generation of large solid, highly vascularized tumors in vivo, which could be detected and measured after 4-5 days.

Treatment of DU-4475 tumor-bearing mice with neutralizing mAb against murine VEGFR-1 (clone MF1) or mAb against murine VEGFR-2 (DC101), 400 μp every three days, to block host-derived angiogenesis, delayed the growth of this breast carcinoma, an effect that was particularly clear between days 14 and 21 post-inoculation. However, this treatment alone was not sufficient to completely block tumor growth, and 21 days after implantation the tumors in MF1 or DC101-treated mice grew to the same size as control (untreated) mice.

Treatment of tumor-bearing mice with neutralizing mAb to human VEGFR-1 (6.12) (400 μg every three days) resulted in a dramatic delay in tumor growth, which was sustained for up to 28 days post- inoculation. Notably, DU-4475-bearing mice did not respond to IMC-IC11 (anti-human VEGFR-2) treatment, confirming these breast tumor cells express only functional VEGFR-1 (Flt-1). However, despite a significant delay in tumor growth, tumors from mice treated with the mAb 6.12 still had viable tumor areas after 21 days. These tumors eventually grew to 1 cm³ after 36 days and started invading the surrounding skin, at which point the mice were sacrificed.

Co-administration of 6.12 (targeting the VEGF/human VEGFR-1 autocrine loop) with MF1 or DC101 (targeting the endothelial-dependent paracrine loop) to DU-4475 bearing mice resulted in a synergistic inhibition of tumor growth. Mice treated simultaneously with 6.12+DC101 (400 μg of each every three days) or 6.12+MF1 (400 μg of each every three days) showed a significant and sustained delay in tumor growth, producing fully necrotic and regressing tumors after 21-28 days, which in the case of the 6.12/DC101 (mAb against human VEGFR-1/mAb against murine VEGFR-2) combination could no longer be measured after 36 days. Therefore, a sustained delay in tumor growth was produced only in mice treated with mAbs against both paracrine and autocrine VEGF/NVEGF receptor signaling pathways.

In vivo Experiments with the DU4475 Breast Cancer Cell Line

Non-obese diabetic immunocompromised (NOD-SCID) mice (Jackson Labs, Bar Harbor, Me., USA) were used in all experiments. DU4475 cells (1×10⁶/mouse) were injected subcutaneously into 21 NOD-SCID mice, and 4 days after injection mice were divided into 7 groups of three mice each. Intraperitoneal treatments started 4 days after cell inoculation. Six of the groups were treated three times a week with the neutralizing mAb: 400 μg of anti-human Flt-1 (clone 6.12), 400 μg of anti-murine VEGFR-1 (mF1), 400 μg of anti-human VEGFR-2 (IMC1-C11), 800 μg of anti-murine VEGFR-2 (DC101). The control group was untreated.

Tumors were measured every 3-4 days for 35 days. When tumors reached approximately 1000 mm³, mice were sacrificed. Tumors were excised, fixed in 2% paraformaldehyde, stored in 70% ethanol and processed for immunohistochemical analysis, following conventional protocols (see above). Paraffin blocks were cut to 5-μm sections and stained with hematoxylin and eosin (H&E), for morphology evaluation.

What is claimed is:

1. A method for preventing or reducing the growth of tumor cells expressing functional VEGF-1 receptors comprising administering to a mammal an effective amount of a VEGF-1 receptor antagonist to inhibit autocrine stimulation.

2. The method of claim 1, wherein the mammal is a human.

3. The method of claim 1, wherein the tumor cells are from a cancer selected from the group consisting of breast cancer, ovarian cancer, brain cancer, kidney cancer, bladder cancer, adenocarcinoma, malignant gliomas and leukemias.

4. The method of claim 3, wherein the cancer is breast cancer.

5. The method of claim 3, wherein the cancer has substantially not vascularized.

6. The method of claim 1, wherein the antagonist is a small molecule.

7. The method of claim 1, wherein the antagonist is an antibody.

8. The method of claim 7, wherein the mammal is a human and the antibody is Mab 6.12, produced by a hybridoma cell line deposited as ATCC number PTA-3344.

9. The method of claim 1, further comprising administering a second antagonist directed to VEGFR expressed on edothelial cells, wherein the VEGFR is selected from the group consisting of VEGFR-2, VEGFR-3 and neuropilin, thereby inhibiting endothelial mediated paracrine loop.

10. The method of claim 9, wherein the second antagonist is a small molecule.

11. The method of claim 9, wherein the second antagonist is an antibody.

12. The method of claim 9, wherein the second antagonist is directed against VEGFR-2.

13. The method of claim 12, wherein the mammal is a human and the antagonist is DC101.

14. The method of claim 1, further comprising administering a chemotherapeutic agent with the antagonist.

15. The method of claim 14, wherein the chmotherapeutic agent is selected from the group consisting of anthracyclin, methotrexate, vindesine, neocarzinostatin, cis-platinum, chlorambucil, cytosine arabinoside, irinotecan, 5-fluorouridine, melphalan, ricin, calicheamicin, taxol, gemcitibine, fluorouracil, paclitaxel, docetaxel, leucovorin and novelbine.

16. The method of claim 1, further comprising administering radiation.
